(19) United States
(12) Patent Application Publication
(10) Pub. No.: US 2004/0062742 A1
(43) Pub. Date: Apr. 1, 2004

(54) Deodorant product containing chlorinating agents and buffered alkaline salts

(76) Inventors: Anthony E. Winston, East Brunswick, NJ (US); Carl G. Mayer, Woodbury Heights, NJ (US)

Correspondence Address: Irving M. Fishman, Suite 1422, North Tower, 89 Headquarters Plaza, Morristown, NJ, 07960, US

(21) Appl. No.: 10/262,208

(22) Filed: Sep. 30, 2002

(51) Int. Cl. A61L 9/00; A61L 9/01

(52) U.S. Cl. 424/76.1

(57) ABSTRACT

A composition comprising a chlorinating agent; a nearly insoluble di- or tri-valent metal salt which is an oxide, a hydroxide, a carbonate, or mixture thereof; and the composition has a pH of less than about 10.5 when in use.

### CROSS-REFERENCE TO RELATED APPLICATIONS

Not Applicable

### STATEMENT REGARDING FEDERALLY SPONSORED RESEARCH OR DEVELOPMENT

Not Applicable

### FIELD OF THE INVENTION

The present invention relates to the field of deodorizers and particularly for deodorizers using chlorinating agents, especially those used where fecal matter is involved. In particular the invention relates to products which, among others, are useful in deodorizing products for ostomy bags, carpet and fabric deodorizers, litter products, toilet deodorizers, bathroom deodorizers, diaper pail deodorizers, etc. The invention further relates to products which also deodorize odors due to short chain aliphatic acids (frequently associated with fecal matter) by a neutralization mechanism. As such, the invention relates to nearly insoluble di and trivalent metal carbonates, oxides, hydroxides and mixtures thereof

### BACKGROUND OF THE INVENTION

Foul odors, due to fecal matter and flatulent discharges, are both unpleasant at best to most people, and embarrassing when they occur in inappropriate places. In some instances these odors are due to inappropriate hygiene, but many times are caused by medical conditions having nothing to do with hygiene. In addition, many places where such odors are encountered are precisely where they are expected to be such as in bathrooms and animal litter boxes. In all of these situations, there has been an ongoing need for suitable products to minimize and/or eliminate the foul odor.

Most of the available literature of fecal odor indicates that the primary malodorant compounds are organic sulfides, methyl mercaptan, and hydrogen sulfide. We have found that chlorinating agents and other oxidizing agents can be used to destroy these compounds or render them non-odiferous by raising the oxidation state of the sulfur atom.

Unfortunately, there are additional components of flatulent malodors that are not sensitive to elimination with these agents. For the most part, these additional malodorous compounds are volatile short chain aliphatic acids. These short chain acids are resistant to oxidation by chlorinating agents, but can be made non-volatile (and therefore non-odoriferous) by neutralization with alkaline materials.

When deodorizing short chain acid components by neutralization, it has been found that generally when the pH of the solution is below about 10.00 or even 10.5, the efficacy and rate of neutralization is low. However, it has also been found that when utilizing chlorinating agents to oxidize the sulfurous malodors, if the pH of the solution used is above about pH 10.00, the oxidizing efficacy of the hypochlorite used drops, perhaps because the concentration of free hypochlorous acid becomes too low. In addition, if the pH is raised above 10.0, the deodorizing solution becomes more irritating to the skin.

As such, it was thought that one could not obtain a suitable single product that would simultaneously and effectively deodorize both sulfurous malodors and short chain acid malodors. Therefore, further improvements in deodorization products for use in connection with fecal and flatulent malodors are desired.

### OBJECTS OF THE INVENTION

It is among the objects of the present invention to provide a deodorizing product which is simultaneously efficacious against both sulfurous malodors and short chain acid malodors.

It is another object of the invention to provide a deodorizing product that is substantially non-irritating to the skin of the user while effective in deodorizing sulfurous and short chain acid malodors.

It is still a further object of the invention to provide a sulfurous malodor and short chain acid malodor deodorant product that can be utilized effectively within a pH range of between about 7.0 and about 10.5.

Still other objects of the invention will be recognized by those of ordinary skill in the art.

### BRIEF SUMMARY OF THE INVENTION

Surprisingly, these objects are achieved by providing a composition comprising a chlorinating agent together with a nearly insoluble di- or tri-valent metal compound, which is an oxide, a hydroxide, a carbonate, or mixture thereof in a composition that has a pH of less than about 10.5 when in use.

### BRIEF DESCRIPTION OF THE DRAWING

Not Applicable

### DETAILED DESCRIPTION OF THE INVENTION

The present invention utilizes a chlorinating agent for elimination or reduction of sulfur containing malodorous compounds; a neutralizer for short chain organic acids which neutralizer is a carbonate, oxide, or hydroxide of a divalent metal or of a trivalent metal and where the neutralizer is nearly insoluble; and the solution in which the other components are contained or the solution which results during the use of the product (during the active deodorization phase) is in the range of from about 7.0 to about 10.5, preferably from 8.0 to 10.0.

Chlorinating Agent

The chlorinating agent for use in the present invention preferably provides a low equilibrium concentration of active hypochlorite, while maintaining a chlorinated reserve which releases additional chlorine as the hypochlorite is utilized. This prevents the deodorizing solution itself from having a strong chlorine odor. Some chlorinating agents useful in the present invention may directly oxidize malodorous compounds without releasing significant concentrations of hypochlorite ion. Generally, one can control the equilibrium concentration of hypochlorite by utilizing chlorinated amines, chlorinated sulfonamides, chlorinated iso-cyanurates, and the like. These materials release small amounts of hypochlorite in aqueous solution. If desired, the levels can be further reduced by incorporating the non-chlorinated counterpart of the chlorinated material mentioned above (e.g. the corresponding non-chlorinated amine in the composition having the chlorinated amine, the non-chlorinated sulfonamide in the composition containing the chlorinated sulfonamide, etc.). This forces the equilibrium in favor of retaining the chlorine bound to the molecule, and away from the release of hypochlorite ion. Alternatively, alkali metal (lithium, sodium or potassium) hypochlorite can be used directly and non-chlorinated amines, sulfonamides, or iso-cyanurates (for example sodium cyanurate) can be added to control the available hypochlorite ion levels. In such systems, much of the chlorine becomes bound to the amine, sulfonamide, and/or iso-cyanurate and the chlorinated derivatives are formed in situ. Such sequestered chlorine will be re-released as hypochlorite is consumed in the oxidation of malodorous compounds. As mentioned above, in some cases the unhydrolysed chlorinating agent may also directly oxidize and deodorize malodorous compounds.

Typically, the level of chlorinating agent used should be equivalent to about 0.05 to about 5% hypochlorite ion, preferably from about 0.1 to about 2%, more preferably about 0.2 to about 1% hypochlorite ion. Specifically suitable chlorinated compounds for use in the present invention include, without limitation, Chloramine-T (sodium salt of N-chloro-4-methylbenzenesulfonamide), Chloramine-B (sodium salt of N-chloro-benzene sulfonamide), sodium salt of N-chloro-paranitrobenzene sulfonamide, trichloromelamine, the sodium or potassium salts of mono- or di-chloromelamine, trichloro-isocyanurate, sodium or potassium mono- or dichlroisocyanurate, the sodium or potassium salts of mono- or dichlorosulfamic acid, monochloro- or 1,3 dichlorohydantoin or its 5,5 alkyl derivatives.

Short Chain Organic Acid Neutralizer

The neutralizing agent for use in the present invention is a nearly insoluble di or trivalent metal compound having an anion selected from carbonate, oxide, and hydroxide which when added to water, does not raise the pH of the water above about 10.5, preferably not above 10.0. Generally the nearly insoluble neutralizing agents have a solubility in water of less than about 0.5%, preferably less than about 0.2% by weight. The solubility of the neutralizing agent in a particular composition may be more or less, the solubility in water (without other components) being the criteria for its suitability in the present invention. Any di or tri valent metal may serve as the cation so long as the compound observes the solubility criteria, the pH criteria, and is compatible with the ultimate use of the formulation in question. Preferred cations include, but are not limited to, calcium, magnesium, aluminum, zinc, and iron. Especially preferred compounds include, but are not limited to, calcium carbonate (e.g. calcite, aragonite, etc.), magnesium carbonate, calcium magnesium carbonate (e.g. dolomite), basic magnesium carbonate, magnesium oxide, magnesium hydroxide, zinc carbonate, zinc oxide, aluminum oxide, ferrous carbonate. A most preferable compound for the neutralizer is calcium carbonate. An alkali metal (sodium or potassium) bicarbonate may be used as the auxiliary component to help buffer the pH so long as the auxiliary component, when added to water does not raise the pH above the levels set out above. The auxiliary component may not necessarily have any effect on short chain acid malodors, but can provide useful adjunct deodorancy and help to buffer the composition in a desirable pH range. Typical levels of the nearly insoluble neutralizer for use in the present invention are in the range of about 0.1% to about 10%, more preferably about 0.5% to about 5%, still more preferably about 1% to about 4% by weight based on the entire formulation.

pH

As stated above, the pH of water to which the neutralizing agent is added is not raised above about 10.5, preferably not raised above 10.0. In addition, the formulation as a whole, prior to use, or if in solid form, then when added to water in an amount which would be reflective of concentrations in the normal intended use, has a pH which is between about 7.0 and about 10.5, preferably between about 8.0 and about 10.0, more preferably between about 8.5 and about 9.5.

Additional Components

Further components that are useful in the present invention include suspending or thickening agents (useful for suspending the neutralizer and maintaining uniformity throughout the suspension of storage); bleach stable fragrance; wetting or cleaning agents; additional neutralizing and deodorizing agents; coloring agent or dye; antimicrobial agents; etc

Suitable thickening agents include xanthan gum, carboxymethyl cellulose, polyvinyl alcohol used at levels between about 0.005% and 1% as well as synthetic or natural bentonite clays used at levels between about 2 and 5%. Particularly suitable are clays which have had the exterior of the particles reacted with polyvalent metal cations. The metal cations are selected from alkaline earth metals and di-, tri-, or tetravalent metal ions, in particular they include, include, without limitation, calcium, magnesium, strontium, barium, zinc, copper, manganese and iron; preferably zinc, copper, and iron (II). Preferably the divalent, trivalent, and tetravalent metal cations are present in an amounts of a salt thereof at about 2×10⁻⁵ moles to 1.67×10⁻³ moles, more preferably 4×10⁻⁵ moles to 8×10⁻⁴ moles of such metal cation per gram of clay. These externally modified clays are more tolerant of the high ionic strengths that may be associated with either the formulations or conditions of use of the formulations. Generally, when the clays are used as thickeners for liquid or semi-liquid/gel formulations, the externally modified clay is prepared by hydrating the clay as a suspension in water and adding the divalent, trivalent, and/or tetravalent metal cation salt to the hydrated clay. The externally reacted clay suspension is then suitable for use as is. Further details concerning these components may be found in the Inventors' copending applications Docket # 49502/000 entitled Pseudo-Plastic Thixotropic Deodorant Product for Ostomy Pouches and Docket #49702/000 entitled High Ionic Strength Tolerant Thickening Systems and Products Formulated Therewith, both filed simultaneously herewith, and incorporated herein by reference.

Wetting agents and cleaning agents include alkyl sulfates and sulfonates with alkyl chain lengths between 8 and 18 and are used at levels between about 0.1 and 1%. Additional deodorizing ingredients include various clays, activated carbon and zeolites used at levels between about 1 and 10%.

Product Forms

The present invention can be in the form of a liquid such as a suspension or emulsion or a solid such as a powder or tablet. When solid forms are desired, deodorizing clays, activated carbon, zeolites, among others, may be used as carriers or adjuncts.

Uses

The present invention formulations can be used in a wide variety of contexts. The main use is in conjunction with removing odors associated with fecal matter and flatulence gasses and include deodorizers for ostomy appliances, as or in conjunction with animal litters, carpet deodorizers or cleaners, especially in conjunction with pet accidents, toilet bowl, bathroom, and commode deodorizers. They are also useful in connection with cleaning and deodorizing barnyard animal stalls; slaughterhouses; bedpans; waste treatment plants; industrial plants where malodorous sulfur containing compounds and/or malodorous short chain acids are produced, particularly as gaseous discharge products); etc.

### EXAMPLES

The following examples exemplify, but do not limit, the present invention.

### Example 1

An ostomy bag deodorizing formulation is formulated as follows:

| | |
|---|---|
| Chloramine - T | 2.0 |
| Calcium Carbonate | 2.0 |
| Sodium bicarbonate | 1.0 |
| Xanthan Gum | 0.05 |
| Laponite D | 4.0 |
| Copper sulfate (3.6%) | 2.4 |
| Fragrance | 0.2 |
| Water | 88.35 |
| Total | 100.00 |

About 2 g of the product is sprayed into an ostomy bag prior to use.

### Example 2

A carpet deodorizer spray for animal accidents is formulated as follows:

| | |
|---|---|
| Sodium hypochlorite | 1.0 |
| Sodium Cyanurate | 5.0 |
| Sodium bicarbonate | 1.0 |
| Basic magnesium carbonate | 1.0 |
| Laponite D | 3.0 |
| Zinc sulfate (4%) | 2.0 |
| Fragrance | 0.5 |
| Sodium Lauryl sulfate | 0.1 |
| Water | 86.4 |
| Total | 100.00 |

The product is applied to the carpet area of the spill and allowed to stand for about 5 minutes. The area is wiped over. For more serious pet accidents, the product can be reapplied and left on the area for a longer period.

### Example 3

A powdered cat litter deodorizer additive is formulated as follows:

| | |
|---|---|
| Chloramine - T | 2.0 |
| Calcium carbonate | 20.0 |
| Zinc oxide | 1.0 |
| Sodium bicarbonate | 1.0 |
| Bentonite Clay | 75.0 |
| Fragrance | 1.0 |
| Total | 100.0 |

The dry product is mixed with the cat litter prior to use.

### Example 4

A latrine or portable toilet deodorizer is formulated as follows:

| | |
|---|---|
| Trichloromelamine | 1.0 |
| Calcium carbonate | 3.0 |
| Dolomite | 95.0 |
| Fragrance | 1.0 |
| Total | 100.0 |

The dry product is sprinkled over the surface after use.

### Example 5

An industrial gaseous malodorant deodorizer is formulated as follows:

| | |
|---|---|
| Chloramine - T | 2.0 |
| Calcium Carbonate | 2.0 |
| Sodium bicarbonate | 1.0 |
| Xanthan Gum | 0.05 |
| Laponite D | 4.0 |
| Copper sulfate (3.6%) | 2.4 |
| Fragrance | 0.2 |
| Water | 88.35 |
| Total | 100.00 |

The composition is sprayed onto a filter material which is placed in the flow path of a gaseous malodorant effluent which gaseous effluent has either or both of sulfur containing gaseous malodorants and/or short chain aliphatic acids. The effluent gas which has passes through the filter treated with the above composition is less odoriferous than the effluent gas prior to contacting the treated filter.

Alternatively or in conjunction with the above filter, the composition above is sprayed onto the one or more walls along the flow path of the effluent gas on its way to being discharged. The effluent gas which is discharged is less odoriferous than effluent gas which has not passed along a flow path having the above composition thereon.

We claim:

1. A neutralizing and chlorinating deodorant composition comprising
(a) a chlorinating agent, for reducing sulfurous compound malodors; and
(b) a short chain organic acid neutralizer, for reducing malodors associated with said short chain organic acid,
said neutralizer being nearly insoluble in water wherein the composition has a usage pH of between 7.0 and 10.5).

2. The composition of claim 1 wherein said chlorinating agent is selected from the group consisting of compounds which result in a low equilibrium concentration of free hypochlorite ion in use.

3. The composition of claim 2 wherein said chlorinating agent is selected from the group consisting of Chloramine-T, Chloramine-B, sodium salt of N-chloro-paranitrobenzene sulfonamide, di chlorosulfamic acid, sodium or potassium mono or dichloroisocyanurate, sodium or potassium mono or di chloromelamine, trichloromelamine, trichloroisocyanurate, monochloro- or 1,3 dichlorohydantoin or its 5,5 dialkyl derivatives and mixtures thereof.

4. The composition of claim 1 wherein said neutralizier is a selected from compounds having a water solubility of less than about 0.5%

5. The composition of claim 4 wherein said neutralizer has a cation selected from the group consisting of divalent, trivalent, and tetravalent metal ions and an anion selected from the group consisting of carbonates, oxides, and hydroxides.

6. The composition of claim 4 wherein said neutralizer, upon being placed in water does not result in a pH of said water in excess of about 10.5

7. The composition of claim 5 wherein said neutralizer has a cation selected from the group consisting of divalent and trivalent metal ions.

8. The neutralizer of claim 5 wherein said neutralizer has a cation selected from the group consisting of calcium, magnesium, zinc, aluminum, and iron.

9. The composition of claim 1 where the usage pH is between 8.0 and 10.0.

10. The composition of claim 1 where the neutralizer is calcium carbonate at a concentration between about 1.0 and about 5.0%.

11. The composition of claim 1 which also contains from 0.5 to 5% sodium bicarbonate.

12. The composition of claim 1 which also contains a chlorine stable fragrance.

13. A method of use of the composition of claim 1 comprising deodorizing odoriferous gases said gases having (a) a sulfur containing odoriferous gaseous component and (b) at least one of (1) an odoriferous gaseous short chain aliphatic acid and (2) an environmentally undesirable gaseous acid component wherein said use is selected from the group consisting of use in or with an ostomy appliance, use as or in conjunction with an animal litter, use as or in a carpet deodorizer or cleaner, use as or in a toilet bowl cleaning or deodorizing product, use as or in a bathroom and/or commode deodorizing product, use as or in industrial deodorizing and/or scrubbing products, use as or in an animal stall cleaning and/or deodorizing product, use as or in a slaughterhouse cleaning and/or deodorizing product, use as or in a bedpans deodorizing and/or cleaning product, use as or in a waste treatment plant deodorizing and/or cleaning product, use as or in an industrial plant deodorizing or cleaning product.
